(19) United States
(12) Patent Application Publication
(10) Pub. No.: US 2015/0283155 A1
(43) Pub. Date: Oct. 8, 2015

(54) FOSFESTROL FOR USE IN CURATIVE OR PALLIATIVE TREATMENT OF PROSTATE CANCER

(71) Applicant: CHAMAELEO PHARMA BVBA, Diegem (BE)

(72) Inventors: Johannes Jan Platteeuw, Diegem (BE); Martijn De Bree, Almere (NL)

(73) Assignee: CHAMAELEO PHARMA BVBA, Diegem (BE)

(21) Appl. No.: 14/435,731

(22) PCT Filed: Oct. 14, 2013
(86) PCT No.: PCT/EP2013/071414

§ 371 Date: Apr. 14, 2015

(30) Foreign Application Priority Data
Oct. 15, 2012 (EP) 12188535.4

(51) Int. Cl. A61K 31/6615

(52) U.S. Cl. CPC A61K 31/6615

(57) ABSTRACT

The present invention relates to the use of Fosfestrol (diethylstilbestrol diphosphate) in a method of curative or palliative treatment of prostate cancer in male mammals, said method comprising orally administering Fosfestrol in a daily dosage of at least 1,000 mg. The inventors have discovered that Fosfestrol when administered in very high oral dosages is effective in the treatment of prostate cancer, especially hormone resistant prostate cancer, without giving rise to serious side effects, such as thromboembolic toxicity or mortality. The invention further provides an oral dosage unit comprising at least 500 mg, of Fosfestrol.

### TECHNICAL FIELD OF THE INVENTION

The present invention relates to the use of Fosfestrol (diethylstilbestrol diphosphate) in curative or palliative treatment of prostate cancer in a male mammal, said treatment comprising oral administration of the Fosfestrol in a daily amount of at least 1,000 mg.

The invention also provides an oral dosage unit containing at least 500 mg of Fosfestrol.

### BACKGROUND OF THE INVENTION

Cancer is still among the major causes of death in the western world. This applies to both males and females. Due to ongoing research on new medicines and methods of treatment, life expectance of people suffering from different types of cancer has steadily increased over the years. Nevertheless, better medicines and enhanced methods of treatment are still needed.

Endocrine treatment essentially adds, blocks, or removes hormones. To slow or stop the growth of certain cancers (such as prostate cancer), synthetic hormones or other drugs may be given to block the body's natural hormones. Sometimes surgery is needed to remove the gland that makes a certain hormone. Endocrine therapy is also known as hormonal therapy, hormone therapy and hormone treatment.

### DES Therapy in Prostate Cancer

Among the medicines that already have been used in the treatment of cancer is diethylstilbestrol (DES). DES is a synthetic nonsteroidal estrogen that was first synthesized in 1938. It was designed to achieve castrate levels of testosterone. Androgens drive prostate cancer growth and withdrawal of androgens by surgical castration was the first androgen ablation therapy in prostate cancer treatment. DES was developed to achieve chemical castration by inhibiting testicular production of androgens.

However, the role of oral administration of DES in the treatment of prostate cancer has been limited because of an association with thromboembolic toxicity. When estrogens like for example DES are given orally, they are subject to the intestinal and hepatic first-pass effect leading to high hormone concentrations in the liver promoting the synthesis of clotting proteins like fibrinogen.

Non-cancer related deaths, mostly cardiovascular in origin, were increased by 36% in patients suffering from prostate cancer receiving 5 mg of DES p.o. per day (Byar D P: Proceedings: The Veterans Administration Cooperative Urological Research Group's studies of cancer of the prostate. Cancer (1973) 32:1126-30). Other studies evaluating lower doses of DES reported similar efficacy towards testosterone suppression as obtained with the 5 mg dose and acceptable thromboembolic toxicity. This led to the adoption of 3 mg per day as the most commonly used DES oral dose for treating prostate cancer. However, the thromboembolic toxicity remained a concern.

DES was replaced as a first line therapy in prostate cancer when a study was published in 1984 by the Leuprolide Study Group comparing the efficacy and safety of 3 mg DES versus Leuprolide in metastatic prostate cancer, which showed similar therapeutic efficacy but a much improved safety profile for Leuprolide (The Leuprolide Study Group (1984) Leuprolide versus diethylstilbestrol for metastatic prostate cancer. N Engl J Med; 311(20):1281-6).

To overcome the objections against high concentrations of DES in the liver, recent patent applications sought ways to administer DES either by transdermal administration of DES (US20030147936A1) or buccal administration (US2011189288A1) thus avoiding the first pass metabolic effect of intestinal enzymes and the liver. Both applicants claimed that by bypassing the liver DES can be safely administered.

In the first case this is achieved by a placing a controlled release implant in the vicinity of the prostate and this implant than releases over an extended period of time an unspecified minute quantity of DES near the target area. No data on the plasma concentrations is available from this publications but they will certainly not be very high.

In the second case the first pass metabolism in the gut and liver are bypassed by buccal administration and adsorption of DES. DES is plasma was detected at levels of on average 11 ng DES/ml, without inducing a thromboembolic activator (Fibrinogen).

### Fosfestrol Therapy

The aforementioned concerns regarding the cardiovascular side effects of DES have led to the development of DES-based formulations that are less prone to intestinal and hepatic first pass effect.

GB 732,286 describes the synthesis of Fosfestrol (diethylstilbestrol diphosphate). Fosfestrol was developed as a prodrug of DES to achieve safe inhibition of testosterone production without causing thromboembolic side effects caused by free DES. The phosphate groups were added to inactivate DES, thereby circumventing the intestinal and hepatic first pass effect and decreasing the circulating levels of free DES. Fosfestrol itself was considered to be inactive and it was known that prostate cancer cells have increased expression of prostate acid phosphatase (PAP). It was thought that PAP would remove the phosphate groups and release DES near its side of action.

With a view to its estrogenic effect on testosterone decrease an oral dose of 200 mg of Fosfestrol is deemed to be equipotent to an oral dose of 3 mg DES, resulting in a similar estrogenic side effect profile.

Fosfestrol was introduced and marketed in the 1950's under the name Honvan® and has been successfully applied in the treatment of prostate cancer for many years. However, as Fosfestrol is a prodrug of DES and DES was associated with problematic side effects it was replaced as a first line therapy at the same time as DES by leuprolide therapy.

Hartley-Asp et al. (Diethylstilbestrol induces metaphase arrest and inhibits microtubule assembly, Mutation Research, 143 (1985) 231-235) investigated the effects of DES on DU-145 prostate cancer cells. They showed cytotoxic effects of DES in the prostate cancer cells through inhibition of microtubule formation.

Oelschläger et al. (New Results on the Pharmacokinetics of Fosfestrol, Urol. Int. 43 (1988), 15-21) have shown that Fosfestrol and its monophosphate exist only for a short time in small amounts in the circulating blood after intravenous administration (1.5 g per day for 10 days), whilst after oral administration (360 mg), not even traces of the phosphates could be detected in the plasma. According to the authors, the most important influence on plasma levels of Fosfestrol and its metabolites is due to the extraction function of the liver. Diethylstilbestrol conjugates enter into the entero-hepatic circle, thus forming a possible source of DES available over more than 24 h.

Schulz et al. (Evaluation of the Cytotoxic Activity of Diethylstilbestrol and Its Mono- and Diphosphate towards Prostatic Carcinoma Cells, Cancer Res 1988; 48:2867-2870) showed that DES concentrations ranging up to 100 ng/ml do not influence prostate cancer cell growth and that the minimal concentration of DES to induce some cytotoxic effects is 1 μg/ml. However as explained before, to achieve such high plasma concentrations via administration of DES or Fosfestrol is commonly associated with unacceptable toxic effects.

The most advanced study towards the use of intravenous Fosfestrol was published by Kattan et al (High dose fosfestrol in phase I-II-trial for the treatment of hormone-resistant prostatic adenocarcinoma, Bull. Cancer 80 (1993), 248-54). Sixteen patients with HRPC were treated by continuous infusion of high dose Fosfestrol according to two schedules: 10 patients were included in a phase I trial of a daily escalating dose from 1.5 g daily to 4.5 g daily for 7 to 10 days. Six other patients were uniformly treated by 4 g/d for 3.5 h for 5 days. Between each course patients received 300 mg/day oral Fosfestrol and 200 mg/d salicylic acid. Of these patients 15 were evaluable, as one patient died on day 3 from tumor progression complicated by an intravascular coagulation disease. There were four objective stabilizations lasting from 2 to 10 months. Subjective improvement of pain was observed in five other patients. There was more than 50% reduction of PSA in eight patients

Orlando et al. (Low-dose continuous oral fosfestrol is highly active in ‘hormone-refractory’ prostate cancer, Annals of Oncology 11 (2000), 177-181) describe the results of a study in which thirty-eight prostate cancer patients with evidence of disease progression after≧2 hormonal treatments (including surgical or chemical orchiectomy and a median of 3 prior treatment lines) were treated with Fosfestrol. Fosfestrol was given orally at an initial dose of 100 mg 3× daily, continuously, until the advent of progressive disease or excessive toxicity. All ongoing hormonal or cytotoxic therapy was discontinued prior to the start of Fosfestrol. In the few patients with a single PSA rise following an initial response, double-dose Fosfestrol was administered. Treatment, as with the initial dose, was again continued until progressive disease (defined as two consecutive rises of PSA over the lowest achieved value), or excessive toxicity. The authors concluded that the degree of activity seen in their series warrants further prospective evaluation of Fosfestrol in this schedule as a single agent and in combination therapy, since it compares favourably in terms of response, survival and symptomatic benefit as well as of toxicity, costs and ease of administration with many other regimens developed for patients with hormone-refractory prostate cancer.

### SUMMARY OF THE INVENTION

The present inventors have found that Fosfestrol (diethylstilbestrol diphosphate), if administered orally in a high daily amount of at least 1,000 mg, can effectively be used in the treatment of prostate cancer, especially in the treatment of hormone resistant sub-types of initially dependent prostate cancer.

Unexpectedly, the inventors have discovered that Fosfestrol, when administered in such high oral dosages, does not give rise to serious side effects, such as thromboembolic toxicity or even mortality.

Without wishing to be bound by theory it is hypothesized that the toxic effects that have been observed in the past for high levels (˜3 mg daily) of orally administered DES are not so much caused by DES itself, but by oxidized DES metabolites.

When DES is given orally it is subjected to intensive intestinal and hepatic metabolism. Metabolisation of orally administered DES occurs through oxidation followed by conjugation or through direct conjugation. The main oxidative reactions are hydroxylation of the aromatic rings and, at the ethyl group, subsequent conjugations. Also formation of a hexadiene has been observed. The conjugates formed are sulphates, or glucuronides or combinations of the two.

The inventors believe that orally administered Fosfestrol is less prone to oxidation than orally administered DES. Thus, the same DES plasma levels can be achieved with orally administered Fosfestrol as with orally administered DES, but with substantially lower levels of oxidized DES metabolites and consequently with significantly less toxic side-effects.

In addition, treatment of prostate cancer using oral administration of Fosfestrol in high doses offers advantages over intravenous administration of high doses of the same substance. More particularly, whereas DES plasma levels tend to drop sharply after discontinuation of intravenous Fosfestrol administration, this is not the case for orally administered Fosfestrol. The inventors believe that this advantage results from the fact that orally administered Fosfestrol is metabolized and that one or more of the metabolites formed act as a ‘reservoir’ for the pharmaceutically active component, i.e. DES, in that they are more gradually converted into DES than intravenously administered Fosfestrol.

The present invention also relates to an oral dosage unit that contains at least 500 mg of Fosfestrol.

### DETAILED DESCRIPTION OF THE INVENTION

A first aspect of the invention concerns Fosfestrol (diethylstilbestrol diphosphate) for use in a method of curative or palliative treatment of prostate cancer in male mammals, said method comprising orally administering Fosfestrol in a daily dosage of at least 1,000 mg.

The term ‘Fosfestrol’ as used herein refers to a diethylstilbestrol moiety of which both the hydroxyl groups are phosphated. The term ‘Fosfestrol’ also encompasses pharmaceutically acceptable salts of Fosfestrol.

The term ‘pharmaceutically acceptable salt’, as used herein, means those salts of compounds of the invention that are safe and effective for use in mammals and that possess the desired biological activity. Descriptions of counter ions for pharmaceutically acceptable salts of pharmaceutical compounds can be found in P. Heinrich Stahl, Camille G. Wermuth (Eds.), Handbook of Pharmaceutical Salts, Properties, Selection and Use, Wiley VCH (2002).

The diethylstilbestrol moiety in the DES phosphate of the present invention may be in the trans-form or the cis-form. Naturally, also mixtures of the trans- and cis-form may be employed.

The term ‘cancer’ as used herein refers to a malignant neoplasm involving unregulated cell growth. In cancer, cells divide and grow uncontrollably, forming malignant tumors, and invade nearby parts of the body.

The term ‘curative treatment’ as used herein refers to a treatment that aims to cure a disease or to improve symptoms associated with a disease.

The term ‘palliative treatment’ as used herein refers to a treatment or therapy that does not aim at curing a disease but rather at providing relief

The term ‘oral’ as used herein, unless indicated otherwise, is synonymous to ‘per oral’.

The term ‘dosage’ as used herein refers to the amount of a pharmaceutically active substance that is administered to a mammal. Hence, the term ‘dosage’ does not include any carrier or other pharmaceutically acceptable excipient that is part of a ‘dosage unit’ to be administered.

In this document and in its claims, the verb ‘to comprise’ and its conjugations are used in their non-limiting sense to mean that items following the word are included, without excluding items not specifically mentioned. In addition, reference to an element by the indefinite article ‘a’ or ‘an’ does not exclude the possibility that more than one of the element is present, unless the context clearly requires that there be one and only one of the elements. The indefinite article ‘a’ or ‘an’ thus usually means ‘at least one’.

Hormone-dependent cancers refer to those types of cancer that grow faster in the presence of particular hormones. This type of cancer is usually treated with hormone therapy. Hormone therapy involves blocking in vivo production or action of these hormones. Therefore, hormone therapy actually is anti-hormone therapy. Cancer of the prostate usually is a hormone-dependent cancer and may be treated by the present method.

In the case of hormone-dependent prostate cancer, androgen ablation therapy (e.g. orchiectomy, treatment with LHRH analogs or LHRH antagonists) is used as first line treatment to decrease the production of androgens, particularly testosterone, in order to stop or limit the growth of prostate cancer. Androgens are key drivers of prostate tumor growth. The androgen ablation therapies reduce the plasma levels of androgen, thereby reducing the growth potential of the prostate tumor. The androgen ablation therapies are successful for a certain period of time, however all prostate tumors eventually become resistant to this treatment approach. After failure of the androgen ablation therapy, secondary hormone treatments with anti-androgens are used to slow the growth of the prostate tumor.

After exposure for a certain time to hormone therapy prostate cancer often obtain the ability to grow without hormones and are therefore called ‘hormone-independent’. Once these cancers become hormone-independent, treatment usually is switched to chemotherapy.

Hormone-independent prostate cancer is also called hormone-refractory or castration-resistant prostate cancer. These terms are used interchangeably in the following and are considered to have the meaning of ‘castration-resistant prostate cancer’. Nowadays, the term ‘castration resistant’ has replaced ‘hormone refractory’ because while these prostate cancers are no longer responsive to castration treatment (reduction of available androgen/testosterone), they still show some reliance upon hormones for androgen receptor activation.

The present invention encompasses the treatment of hormone-dependent as well as hormone-independent cancers. The present method is particularly suited for treatment of hormone-independent cancers, especially for treatment of hormone-independent cancers that have developed after treatment of hormone dependent cancers with hormone therapy.

The present method of treatment is advantageously applied to treat a prostate cancer that does not respond to treatment with anti-androgen or an inhibitor of 17α hydroxylase/C17,20 lyase (CYP17A1), especially a prostate cancer that does not respond to treatment with an inhibitor of 17α hydroxylase/C17,20 lyase (CYP17A1), more particularly to treatment with Abiraterone. The present method is particularly suited for treatment of hormone-independent prostate cancer that has developed after treatment of hormone-dependent prostate cancer with anti-androgen or an inhibitor of 17α hydroxylase/C17,20 lyase (CYP17A1), notably Abiraterone.

As explained herein before, Fosfestrol in the context of the present invention also encompasses pharmaceutically acceptable salts of Fosfestrol. Pharmaceutically acceptable salts include those formed from cations of alkali metals such as sodium, lithium, potassium, and earth alkali metals such as calcium and magnesium.

In a preferred embodiment the Fosfestrol is an alkali metal salt, notably a sodium and/or a potassium salt. More preferably, the Fosfestrol is in the potassium salt form.

The present method of treatment may be used to treat several kinds of mammals, e.g. humans, horses, cattle etc. The present method is particularly suited for the treatment of humans.

The Fosfestrol dosage may vary depending upon the specific conditions and patients undergoing treatment. The therapeutically effective dosage of the compound can be provided as repeated doses within a prolonged treatment regimen that will yield clinically significant results.

The actual dosage of the compound will vary according to factors such as the disease indication and particular status of the subject such as for example, age, size, fitness, extent of symptoms, susceptibility factors and the like, and other factors such as time and route of administration, other drugs or treatments being administered concurrently. Dosage regimens can be adjusted to provide an optimum therapeutic response.

Typically, the present method comprises administering Fosfestrol in a daily oral amount of at least 1,000 mg, more preferably of 1,000-4,500 mg and most preferably of 1,000-2,000 mg.

Expressed differently, it is preferred to administer Fosfestrol orally in a daily amount of at least 12.5 mg per kg of bodyweight, more preferably of 12.5-60 mg per kg of bodyweight and most preferably of 12.5-27 mg per kg of bodyweight.

The duration of the present method of treatment typically exceeds 7 days. More particularly, the present method has a duration of at least 14 days, especially of at least 28 days.

The aforementioned daily amount may be administered once daily of it may be administered in the form of two or more separate doses at more or less regular intervals. According to a particularly preferred embodiment, the present method of treatment comprises orally administering at least two doses per day, more preferably two doses of each at least 200 mg Fosfestrol per day, even more preferably it comprises orally administering at least 3 doses of at least 200 mg Fosfestrol per day.

Another aspect of the invention relates to an oral dosage unit comprising at least 500 mg, preferably at least 800 mg and most preferably at least 1,000 mg, of Fosfestrol.

The oral dosage unit of the present invention can advantageously be applied in the curative or palliative treatment of prostate cancer as defined herein before.

The oral dosage units is preferably selected from the group consisting of tablets, granulates, capsules and powders and liquids. Even more preferably, the oral dosage unit is a tablet or capsule.

The oral dosage units typically have a weight of between 0.5 and 2.0 g, more preferably of 0.75-1.5 g and most preferably of 0.8-1.2 g. In another embodiment, the oral dosage units comprise between 20 and 80 wt. % of pharmaceutically acceptable excipient. The pharmaceutically acceptable excipient is suitably selected from coloring agents, flavoring or taste masking agents, diluents, binders, lubricants, disintegrants, stabilizers, surfactants, glidants, plasticizers, preservatives, sweeteners and combinations thereof.

The disintegrants are advantageously chosen from the group consisting of lactose, anhydrous lactose, crospovidone, croscarmellose sodium, sodium starch glycolate, hydroxypropyl cellulose, polacrilin potassium, pregelatinized starch, microcrystalline cellulose and combinations thereof. In a preferred embodiment the oral dosage units comprise up to 7 wt. %, preferably 2-5 wt. % of disintegrants.

The dosage unit of the present invention may suitably take the shape of a compressed tablet. Such a tablet may suitably comprise two or more layers of different composition, for example a core comprising Fosfestrol as defined herein before encased in a coating.

The dosage units of the present inventions are conveniently produced in a tabletting machine. In order to enable easy removal of the tablets from the moulds, the dosage unit typically contains between 0.2 and 4.0 wt. % of a lubricant or gliding agent. Preferably, the lubricant or gliding agent is selected from the group consisting of talc, sodium stearyl fumarate, magnesium stearate, calcium stearate, hydrogenated castor oil, hydrogenated soybean oil, polyethylene glycol, starches, anhydrous colloidal silica and combinations thereof.

The following examples are meant to further illustrate the invention and some of its preferred embodiments without intending to limit its scope.

### EXAMPLES

### Example 1

The in vitro direct cytotoxicity of DES and Fosfestrol in hormone-dependent (LNCaP) and hormone-independent (DU-145) prostate cancer cell lines was tested.

Cells were maintained in vitro in RPMI 1640 containing 10% (v/v) heat inactivated fetal bovine serum (FBS) and 2 mM L-glutamine (growth media) at 37° C. in 5% CO₂ and humidified conditions. Cells were harvested, washed, re-suspended into growth medium and counted. The cells were re-suspended into assay media (RPMI 1640+1% (v/v) heat inactivated FBS+ and 2 mM L-glutamine) at 0.5×10⁵ cells/ml for DU-145 cells and 1×10⁵ for LNCaP cells, and plated into 96-well assay plates (Corning, black-wall plates) and 50 μl/well aliquots.

Plates were incubated O/N at 37° C. in 5% humidified CO₂ prior to addition of the compounds. DES was dissolved in 100% DMSO at stock concentration of 60 mM. Fosfestrol was dissolved in sterile water at stock concentration of 60 mM. Stocks of all compounds were then serially diluted. Final concentrations to which cells were exposed were: 300, 150, 75, 37.5, 18.75, 9.4, 4.7, 2.3, 1.2 and 0.6 μM. Positive control was Taxotere. Taxotere was diluted in 100% DSMO to give a stock concentration of 1 mM. Stock was serial diluted and final concentration to which cells were exposed was: 1000, 333.3, 111.1, 37.0, 12.3, 4.1, 1.4, 0.5, and 0.2 nM.

Plates were incubated for 72 hrs at 37° C. in 5% humidified CO-2 after addition of the compounds. Viability of the cells was assessed with the Cell titer blue® (Promega) assay. 10 μl of Cell titer Blue™ reagents was added to each test/blank well. Plates were incubated for 3 hrs at 37° C. in 5% humidified CO₂ prior to analysis. Fluorescence was measured with a Flex II station plate reader. Excitation wavelength was 570 nm, emission wave length was 600 nm, cut off was 590 nm. Raw data was processed by GraphPad Prism to calculate mean, standard deviation and IC₅₀ values.

The results so obtained are shown in Table 1.

TABLE 1

IC₅₀ value (μM)

| | LNCaP | DU-145 |
|---|---|---|
| DES | 27 | 62 |
| Fosfestrol | 70 | 84 |
| Taxotere (control) | 0.002 | 0.004 |

### Conclusion:

These results show that DES and Fosfestrol are both cytotoxic in LNCaP and DU-145 prostate cancer cells.

### Example 2

A 1 kg batch of 500 mg Fosfestrol tablets was prepared by direct compression as described below.

Fosfestrol and excipients were first passed over a 0.85 mm sieve. Next, 500 gram of fosfestrol tetrasodium was blended with 435 gram of silicified Microcrystalline Cellulose (Prosolv smcc 90™) and 50 gram of croscarmellose sodium (Ac-di-Sol™) for 20 minutes in a V-blender. Added to the mixture was 15 grams of magnesium stearate and blending was continued for 5 minutes.

Tablets of 1,000 mg each were prepared on a Korsch EKO, using oval punches.

### Example 3

A patient study was conducted in chemo and hormone resistant prostate cancer patients to explore the effects of high dose oral Fosfestrol treatment.

11 patients were included into the study and all had undergone at least 2 prior treatments (mostly Taxotere and Estramustine) with a maximum of prior 4 treatments.

Patients were treated with three times 360 mg/d oral Fosfestrol for 4 weeks. Total Fosfestrol dose per day was 1,080 mg. All other treatments were stopped during high dose oral Fosfestrol therapy.

PSA decline was used to measure objective response. 72% of the patients showed a PSA decline of>50% during high dose oral Fosfestrol treatment. In addition, 54% of the patients experienced a>80% PSA decline.

Treatment was accompanied by minor toxicities and no thromboembolic side effects were detected.

### Conclusion:

This study showed that high dose oral Fosfestrol is effective and safe to use in heavily pretreated chemo and hormone resistant prostate cancer patients.

1-13. (canceled)

14. A method of curative or palliative treatment of prostate cancer in male mammals, comprising orally administering a daily amount of at least 1,000 mg fosfestrol (diethylstilbestrol diphosphate).

15. The method according to claim 14, wherein the method comprises orally administering a daily amount of 1,000-4,500 mg fosfestrol.

16. The method according to claim 14, wherein the fosfestrol is administered daily for at least 7 days.

17. The method according to claim 14, wherein the method comprises orally administering at least 12.5 mg of fosfestrol per kg of bodyweight.

18. The method according to claim 14, wherein the method comprises at least twice daily oral administration of fosfestrol.

19. The method according to claim 14, wherein the prostate cancer is castrate-resistant prostate cancer.

20. The method according to claim 19, wherein the castrate-resistant prostate cancer has developed after treatment with anti-androgen or an inhibitor of 17α hydroxylase/C17,20 lyase (CYP17A1).

21. The method according to claim 20, wherein the castrate-resistant prostate cancer has developed after treatment with Abiraterone.

22. The method according to claim 14, wherein the mammal is a human.

23. An oral dosage unit comprising at least 500 mg of fosfestrol.

24. The oral dosage unit according to claim 23, in the form of a tablet or capsule.

25. The oral dosage unit according to claim 23, having a weight of 0.5-2.0 g.

26. The oral dosage unit according to claim 23, comprising 20-80 wt. % of one or more pharmaceutically acceptable excipients.
